(19) United States
(12) Patent Application Publication
(10) Pub. No.: US 2023/0026404 A1
(43) Pub. Date: Jan. 26, 2023

(54) WEARABLE MEDICAL DEVICES DETECTING DECLINES IN HEALTH AND PROBABILITY OF ADVERSE EVENTS

(71) Applicant: West Affum Holdings DAC, Dublin (IE)

(72) Inventor: Mark Paul Moore, Redmond, WA (US)

(73) Assignee: West Affum Holdings DAC, Dublin (IE)

(21) Appl. No.: 17/871,744

(22) Filed: Jul. 22, 2022

Related U.S. Application Data
(60) Provisional application No. 63/224,747, filed on Jul. 22, 2021.

(51) Int. Cl. A61N 1/39; G16H 50/30; G16H 20/70

(52) U.S. Cl. CPC A61N 1/3904; A61N 1/3993; G16H 50/30; G16H 20/70

(57) ABSTRACT

Disclosed are systems and methods that implement a reporting function to capture subjective a subjective health assessment of the patient and to correlate that subjective health assessment with more objective patient parameter data, such as ECG waveforms and the like, in an attempt to identify nuances in the objective patient parameter data that can be used to predict future (or even imminent) adverse health events.

### CROSS-REFERENCE TO RELATED APPLICATIONS

The present application claims priority to and the benefit of U.S. Provisional Application No. 63/224,747, filed Jul. 22, 2021, the entire disclosure of which is hereby incorporated by reference herein for all purposes.

### SUMMARY OF THE DISCLOSURE WITH BACKGROUND

When people suffer from some types of heart arrhythmias, the result may be that blood flow to various parts of the body is reduced. Some arrhythmias may even result in Sudden Cardiac Arrest (SCA). SCA can lead to death very quickly unless treated, e.g., within 10 minutes. Some observers mistake SCA for a heart attack, which it is not.

Some people have an increased risk of SCA. Such people include patients who have had a heart attack or a prior SCA episode. A frequent recommendation for these people is to receive an Implantable Cardioverter Defibrillator (ICD). The ICD is surgically implanted in the chest, and continuously monitors the patient's electrical activity. If certain heart arrhythmias are detected, the ICD delivers an electric shock directly to the heart in an attempt to correct the arrhythmia.

As a further precaution, people who have been identified with an increased risk of SCA are sometimes given a Wearable Cardioverter Defibrillator (WCD) system, to wear until their ICD is implanted, or until their cardiac condition no longer puts them at high risk of SCA. A WCD system typically includes a support structure, such as a harness, vest, belt, or other garment that the patient is to wear. The WCD system further includes electronic components, such as a defibrillator and electrodes, coupled to the support structure. When the patient wears the WCD system, the electrodes make electrical contact with the patient's skin, and therefore can help sense the patient's electrocardiogram (ECG). If a shockable heart arrhythmia (e.g., ventricular fibrillation (VF) or ventricular tachycardia (VT)) is detected from the ECG, the defibrillator delivers an appropriate electric shock through the patient's body, and thus through the heart. The delivered shock may restart the patient's heart and thus save the patient's life.

Remote monitoring is a rapidly growing field of patient care. As such, devices, such as a WCD system with monitoring capabilities and connectivity to a remote monitoring product, provide a clinical user with the ability to view data acquired by the device. However, in most cases, these clinical users are without any subjective contextual information that can be used to assess whether a patient wearing a WCD system is in a steady state of health or is in a declining state of health. In other words, conventional physiological information about the patient collected by the WCD system only reveals whether the patient is well; it cannot reveal whether the patient feels well. Often, a patient about to experience some form of health event, such as SCA, may actually experience subjective feelings of general unhealth prior to the event even though the patient's ordinary physiological parameters do not give a clear indication that such event is imminent.

Embodiments of this disclosure implement a reporting function to capture subjective a subjective health assessment of the patient and to correlate that subjective health assessment with more objective patient parameter data, such as ECG waveforms and the like, in an attempt to identify nuances in the objective patient parameter data that can be used to predict future (or even imminent) adverse health events.

The disclosed embodiments can be advantageous over conventional systems. For example, patients may detect deterioration of their health prior to a medical device detecting significant changes in vital signs or signals. Capturing the patient's assessment of how they feel (patient's own health rating) and providing that information to clinicians, such as the patient's physician or other care giver, may be useful for recognizing or predicting significant declines in health or adverse events (SCD, AMI, Stroke, TIA, etc.).

None of the subject matter discussed in this section is necessarily prior art and may not be presumed to be prior art simply because it is presented in this section. Any reference to any prior art in this description is not, and should not be taken as, an acknowledgment or any form of suggestion that such prior art forms parts of the common general knowledge in any art in any country. Along these lines, any recognition of problems in the prior art discussed in this section or associated with such subject matter should not be treated as prior art, unless expressly stated to be prior art. Rather, the discussion of any subject matter in this section should be treated as part of the approach taken towards solving the particular problems identified. This approach in and of itself may also be inventive.

The summary provided above is intended to introduce a selection of concepts in a simplified form that are further described below in the Detailed Description. This summary is not intended to identify key features of the claimed subject matter, nor is it intended to be used as an aid in determining the scope of the claimed subject matter.

### BRIEF DESCRIPTION OF THE DRAWINGS

Embodiments of the disclosure are best illustrated by way of example and not by way of limitation in the figures of the accompanying drawings, briefly described below, in which like reference numerals indicate similar elements.

FIG. 1 is a conceptual diagram of a patient wearing an exemplary WCD, made according to embodiments.

FIG. 2 is a conceptual diagram of sample embodiments of components of a WCD system made in accordance with this disclosure.

FIG. 3 is a functional diagram showing components of an illustrative external defibrillator made according to embodiments.

FIG. 4 is a functional diagram showing components of an illustrative mobile medical device made according to embodiments.

FIG. 5 is a conceptual diagram generally illustrating a health monitoring environment according to embodiments.

FIG. 6 is a functional flow diagram implementing a process for receiving direct input from a patient, in accordance with the disclosure.

FIG. 7 is a functional flow diagram implementing a process for receiving passive input from a patient, in accordance with the disclosure.

FIG. 8 is a flow diagram generally illustrating another form of passive input embodiment that analyzes a patient's gait to determine a health assessment rating.

FIG. 9 is a functional flow diagram generally illustrating an enhanced process that may be implemented in systems that have a gyroscope, in addition to one or more accelerometers, and are configured to detect patient falls.

### DETAILED DESCRIPTION

Generally described, this disclosure is directed at a system to enhance the prediction of adverse events by correlating a subjective health assessment for a patient with objective patient parameter data and analyzing those data. While illustrative embodiments are described, it will be appreciated that various changes can be made therein without departing from the spirit and scope of the disclosure. It should also be noted that references to “an embodiment” or “one embodiment” in this disclosure are not necessarily to the same embodiment, and those terms mean at least but not necessarily one.

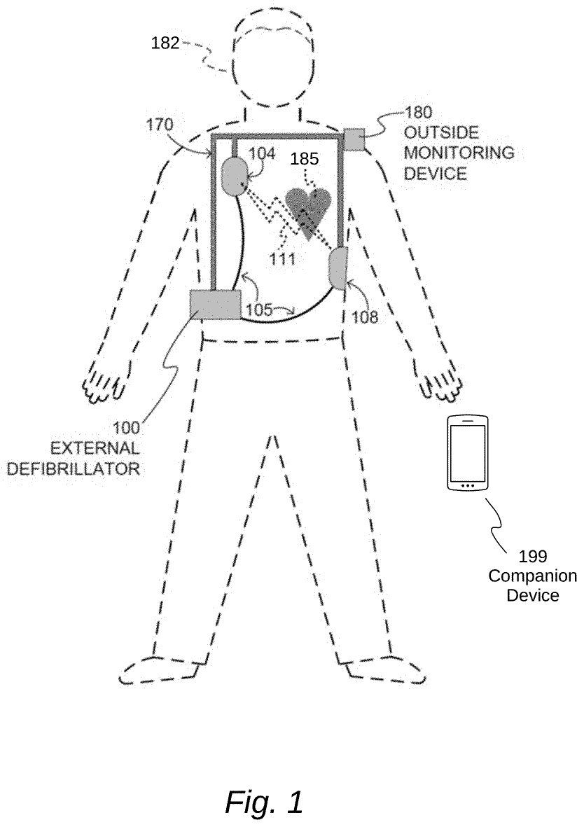

Turning now to the drawings, FIG. 1 depicts a Wearable Cardioverter Defibrillator (WCD) system being worn by a patient 182, according to embodiments of the disclosure. The WCD described herein is presented as one example of a cardiac monitoring system or device that measures and captures cardiac data (e.g., ECG trace data) for a patient wearing the cardiac monitoring system.

Patient 182 may also be referred to as a person and/or wearer since the patient is wearing components of the WCD system. As shown, patient 182 is ambulatory, which means that while wearing the wearable portion of the WCD system under ordinary circumstances, patient 182 can walk around and is not necessarily bed ridden. While patient 182 may be considered to be also a “user” of the WCD system, this is not a requirement. For instance, a user of the wearable cardioverter defibrillator (WCD) may also be a clinician such as a doctor, nurse, emergency medical technician (EMT) or other similarly tasked individual or group of individuals. In some cases, a user may even be a bystander. The particular context of these and other related terms within this description should be interpreted accordingly.

A WCD system according to embodiments can be configured to defibrillate the patient who is wearing the designated parts of the WCD system. Defibrillating can be by the WCD system delivering an electrical charge to the patient's body in the form of an electric shock. The electric shock can be delivered in one or more pulses and/or again should the WCD continue to detect a shockable rhythm.

In particular, FIG. 1 also depicts components of a WCD system made according to embodiments. One such component is a support structure 170 that is wearable by ambulatory patient 182. Accordingly, support structure 170 is configured to be worn by ambulatory patient 182 for at least several hours per day, and for at least several days, even a few months. It will be understood that support structure 170 is shown only generically in FIG. 1, and in fact partly conceptually. FIG. 1 is provided merely to illustrate concepts about support structure 170 and is not to be construed as limiting how support structure 170 is implemented, or how it is worn.

Support structure 170 can be implemented in many different ways. For example, it can be implemented in a single component or a combination of multiple components. In embodiments, support structure 170 could include a vest, a half-vest, a garment, etc. In such embodiments such items can be worn similarly to analogous articles of clothing. In embodiments, support structure 170 could include a harness, one or more belts or straps, etc. In such embodiments, such items can be worn by the patient around the torso, hips, over the shoulder, etc. In embodiments, support structure 170 can include a container or housing, which can even be waterproof. In such embodiments, the support structure can be worn by being attached to the patient's body by adhesive material, for example as shown and described in U.S. Pat. No. 8,024,037. Support structure 170 can even be implemented as described for the support structure of US Pat. App. No. US2017/0056682, which is incorporated herein by reference. Of course, in such embodiments, the person skilled in the art will recognize that additional components of the WCD system can be in the housing of a support structure instead of being attached externally to the support structure, for example as described in the US2017/0056682 document. There can be other examples.

FIG. 1 shows a sample external defibrillator 100. As described in more detail later in this document, some aspects of external defibrillator 100 include a housing and an energy storage module within the housing. As such, in the context of a WCD system, defibrillator 100 is sometimes called a main electronics module. The energy storage module can be configured to store an electrical charge. Other components can cause at least some of the stored electrical charge to be discharged via electrodes through the patient, so as to deliver one or more defibrillation shocks through the patient.

FIG. 1 also shows sample defibrillation electrodes 104, 108, which are coupled to external defibrillator 100 via electrode leads 105. Defibrillation electrodes 104, 108 can be configured to be worn by patient 182 in a number of ways. For instance, defibrillator 100 and defibrillation electrodes 104, 108 can be coupled to support structure 170, directly or indirectly. In other words, support structure 170 can be configured to be worn by ambulatory patient 182 so as to maintain at least one of electrodes 104, 108 on the body of ambulatory patient 182, while patient 182 is moving around, etc. The electrode can be thus maintained on the body by being attached to the skin of patient 182, simply pressed against the skin directly or through garments, etc. In some embodiments the electrode is not necessarily pressed against the skin but becomes biased that way upon sensing a condition that could merit intervention by the WCD system. In addition, many of the components of defibrillator 100 can be considered coupled to support structure 170 directly, or indirectly via at least one of defibrillation electrodes 104, 108.

When defibrillation electrodes 104, 108 make good electrical contact with the body of patient 182, defibrillator 100 can administer, via electrodes 104, 108, a brief, strong electric pulse 111 through the body. Pulse 111 is also known as shock, defibrillation shock, therapy, electrotherapy, therapy shock, etc. Pulse 111 is intended to go through and restart heart 185, in an effort to save the life of patient 182. Pulse 111 can further include one or more pacing pulses of lesser magnitude to simply pace heart 185 if needed, and so on.

Conventional defibrillators typically decide whether to defibrillate or not based on an ECG signal of the patient. However, external defibrillator 100 may initiate defibrillation, or hold-off defibrillation, based on a variety of inputs, with the ECG signal merely being one of these inputs.

A WCD system according to embodiments can obtain data from patient 182. For collecting such data, the WCD system may optionally include at least an outside monitoring device 180. Device 180 is called an “outside” device because it could be provided as a standalone device, for example not within the housing of defibrillator 100. Device 180 can be configured to sense or monitor at least one local parameter. A local parameter can be a parameter of patient 182, or a parameter of the WCD system, or a parameter of the environment, as will be described later in this document.

For some of these parameters, monitoring device 180 may include one or more sensors or transducers. Each one of such sensors can be configured to sense a parameter of patient 182, and to render an input responsive to the sensed parameter. In some embodiments the input is quantitative, such as values of a sensed parameter; in other embodiments the input is qualitative, such as informing whether or not a threshold is crossed, and so on. Sometimes these inputs about patient 182 are also referred to herein as physiological inputs and patient inputs. In embodiments, a sensor can be construed more broadly, as encompassing many individual sensors.

Optionally, monitoring device 180 is physically coupled to support structure 170. In addition, monitoring device 180 may be communicatively coupled with other components that are coupled to support structure 170. Such communication can be implemented by a communication module, as will be deemed applicable by a person skilled in the art in view of this description.

In embodiments, one or more of the components of the shown WCD system may be customized for patient 182. This customization may include a number of aspects. For instance, support structure 170 can be fitted to the body of patient 182. For another instance, baseline physiological parameters of patient 182 can be measured, such as the heart rate of patient 182 while resting, while walking, motion detector outputs while walking, etc. The measured values of such baseline physiological parameters can be used to customize the WCD system, in order to make its diagnoses more accurate, since patients' bodies differ from one another. Of course, such parameter values can be stored in a memory of the WCD system, and so on. Moreover, a programming interface can be made according to embodiments, which receives such measured values of baseline physiological parameters. Such a programming interface may input automatically in the WCD system these, along with other data.

WCD system may further include a “companion” device 199. In various embodiments, the companion device 199 may be implemented as a mobile medical device that also includes various sensors for capturing patient parameters and/or environmental parameters. For example, the companion device 199 may include motion detection sensors, accelerometers, gyroscopic sensors, GPS location sensors, and the like. The companion device 199 further includes a user interface that enables the patient 182 to provide input to and receive output from the companion device 199.

In the preferred embodiment, the companion device 199 is in communication with either the external defibrillator 100, the outside monitoring device 180 (if present), or both. Similarly, the companion device 199 may be in wireless communication with remote computing systems over a local or wide area network. For example, in various embodiments the companion device 199 may be implemented as a special purpose mobile communication device or as a downloadable app that may be installed on a cellular smartphone, or the like.

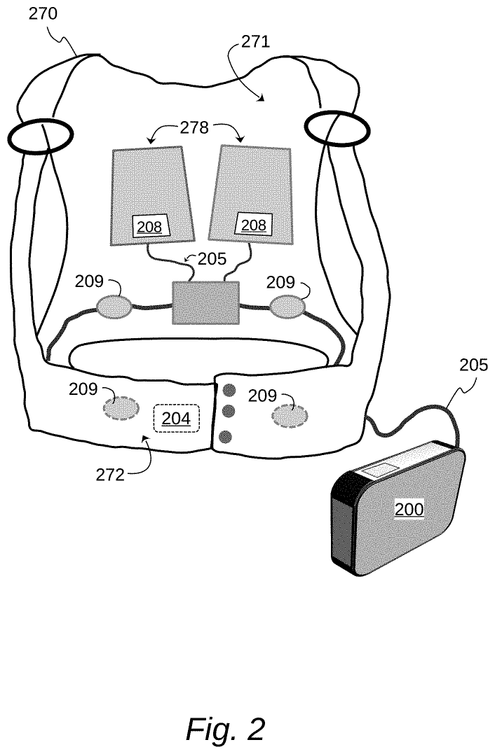

FIG. 2 is a conceptual diagram of components of an illustrative WCD system. A support structure 270 includes a vest-like wearable garment. Support structure 270 has a back side 271, and a front side 272 that closes in front of the chest of the patient.

The WCD system of FIG. 2 also includes an external defibrillator 200. FIG. 2 does not show any support for external defibrillator 200, which may be carried in a purse, on a belt, by a strap over the shoulder, and so on. Wires 205 connect external defibrillator 200 to electrodes 204, 208, 209. Of those, electrodes 204, 208 are defibrillation electrodes, and electrodes 209 are ECG sensing electrodes. The electrodes shown on the front side 272 of the support structure 270 are illustrated in dashed line to represent that those electrodes are within the support structure 270 so that the electrodes may contact the patient.

Support structure 270 is configured to be worn by the ambulatory patient so as to maintain electrodes 204, 208, 209 in contact with the body of the patient. Back defibrillation electrodes 208 may be maintained in pockets of the support structure 270. Of course, the inside of pockets 278 can be made with conductive fabric, so that electrodes 208 can contact the back of the patient, especially with the help of conductive fluid that may be deployed. In addition, sensing electrodes 209 are maintained in positions that surround the patient's torso, for sensing ECG signals and/or the impedance of the patient.

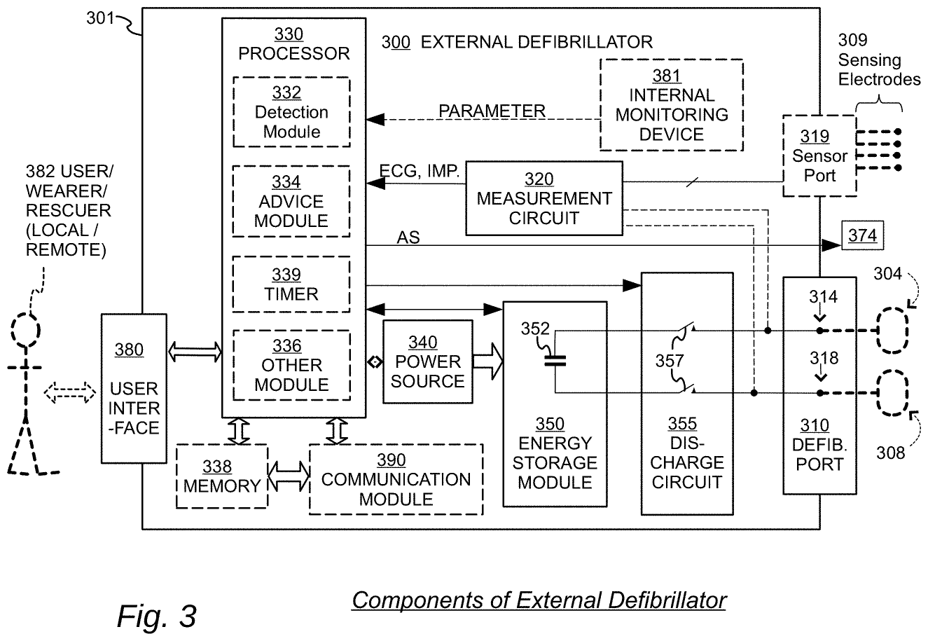

FIG. 3 is a diagram showing certain components of an illustrative external defibrillator 300, made according to embodiments. These components can be, for example, included in external defibrillator 100 of FIG. 1 and external defibrillator 200 of FIG. 2. External defibrillator 300 is intended for a patient who would be wearing it, such as ambulatory patient 182 of FIG. 1. The components shown in FIG. 3 are illustrative, and additional components not shown may, of course, be included.

The components shown in FIG. 3 can be provided in a housing 301, which may also be referred to as a casing. Defibrillator 300 may further include a user interface 380 for a user 382. User 382 can be patient 182, also known as wearer 182. Alternatively, user 382 can be a local rescuer at the scene, such as a bystander who might offer assistance, or a trained person. Or user 382 might be a remotely located trained caregiver in communication with the WCD system.

User interface 380 can be made in a number of ways. User interface 380 may include output devices, which can be visual, audible, or tactile, for communicating to a user by outputting images, sounds or vibrations. Images, sounds, vibrations, and anything that can be perceived by user 382 can also be called human-perceptible indications (HPIs). There are many examples of output devices. For example, an output device can be a light, or a screen to display what is sensed, detected and/or measured, and provide visual feedback to rescuer 382 for their resuscitation attempts, and so on. Another output device can be a speaker, which can be configured to issue voice prompts, beeps, loud alarm sounds and/or words to warn bystanders, etc.

User interface 380 may further include input devices for receiving inputs from users. Such input devices may include various controls, such as pushbuttons, keyboards, touchscreens, one or more microphones, and so on. An input device can be a cancel switch, which is sometimes called an “I am alive” switch or “live man” switch. In some embodiments, actuating the cancel switch can prevent the impending delivery of a shock.

Defibrillator 300 may include an internal monitoring device 381. Device 381 is called an “internal” device because it is incorporated within housing 301. Monitoring device 381 can sense or monitor patient parameters such as patient physiological parameters, system parameters and/or environmental parameters, all of which can be called patient data. In other words, internal monitoring device 381 can be complementary or an alternative to outside monitoring device 180 of FIG. 1. Allocating which of the parameters are to be monitored by which of monitoring devices 180, 381 can be done according to design considerations. Device 381 may include one or more sensors, as also described elsewhere in this document.

Patient parameters may include patient physiological parameters. Patient physiological parameters may include, for example and without limitation, those physiological parameters that can be of any help in detecting by the WCD system whether or not the patient is in need of a shock or other intervention or assistance. Patient physiological parameters may also optionally include the patient's medical history, event history and so on. Examples of patient parameters include the patient's ECG, blood oxygen level, blood flow, blood pressure, blood perfusion, pulsatile change in light transmission or reflection properties of perfused tissue, heart sounds, heart wall motion, breathing sounds and pulse. Accordingly, monitoring devices 180, 381 may include one or more sensors (described below) configured to acquire patient physiological signals.

Patient state parameters include recorded aspects of patient 382, such as motion, posture, whether they have spoken recently plus maybe also what they said, and so on, plus optionally the history of these parameters. Or, one of these monitoring devices could include a location sensor such as a Global Positioning System (GPS) location sensor. Such a sensor can detect the location, plus a speed can be detected as a rate of change of location over time. Many motion detectors output a motion signal that is indicative of the motion of the detector, and thus of the patient's body. Patient state parameters can be very helpful in narrowing down the determination of whether SCA is indeed taking place.

In some embodiments, a trend may be detected in a monitored physiological parameter of patient 382. A trend can be detected by comparing values of parameters at different times over short and long terms. Parameters whose detected trends can particularly help include: (a) cardiac function (e.g., ejection fraction, stroke volume, cardiac output, etc.); (b) heart rate variability at rest or during exercise; (c) heart rate profile during exercise and measurement of activity vigor, such as from the profile of an accelerometer signal and informed from adaptive rate pacemaker technology; (d) heart rate trending; (e) perfusion, such as from SpO2, CO2, or other parameters such as those mentioned above, (f) respiratory function, respiratory rate, etc.; (g) motion, level of activity; and so on. Once a trend is detected, it can be stored and/or reported via a communication link, along perhaps with a warning if warranted. From the report, a physician monitoring the progress of patient 382 will know about a condition that is either not improving or deteriorating.

A WCD system made according to embodiments may include a motion detector. In embodiments, a motion detector can be implemented within monitoring device 180 or monitoring device 381. Such a motion detector can be made in many ways as is known in the art, for example by using an accelerometer. In this example, a motion detector 387 is implemented within monitoring device 381. A motion detector of a WCD system according to embodiments can be configured to detect a motion event. A motion event can be defined as is convenient, for example a change in motion from a baseline motion or rest, etc. In such cases, a sensed patient parameter is motion.

System parameters of a WCD system can include system identification, battery status, system date and time, reports of self-testing, records of data entered, records of episodes and intervention, and so on. In response to the detected motion event, the motion detector may render or generate, from the detected motion event or motion, a motion detection input that can be received by a subsequent device or functionality.

Environmental parameters can include ambient temperature and pressure. Moreover, a humidity sensor may provide information as to whether or not it is likely raining Presumed patient location could also be considered an environmental parameter. The patient location could be presumed, if monitoring device 180 or 381 includes a GPS location sensor as per the above, and if it is presumed that the patient is wearing the WCD system.

Defibrillator 300 typically includes a defibrillation port 310, which can be a socket in housing 301. Defibrillation port 310 includes electrical nodes 314, 318. Leads of defibrillation electrodes 304, 308, such as leads 105 of FIG. 1, can be plugged into defibrillation port 310, so as to make electrical contact with nodes 314, 318, respectively. It is also possible that defibrillation electrodes 304, 308 are connected continuously to defibrillation port 310, instead. Either way, defibrillation port 310 can be used for guiding, via electrodes, to the wearer at least some of the electrical charge that has been stored in an energy storage module 350 that is described more fully later in this document. The electric charge will be the shock for defibrillation, pacing, and so on.

Defibrillator 300 may optionally also have a sensor port 319 in housing 301. Commonly, but not exclusively, the sensor port 319 may be implemented as an ECG port. Sensor port 319 can be adapted for plugging in sensing electrodes 309, which may include ECG electrodes and ECG leads. It is also possible that sensing electrodes 309 can be connected continuously to sensor port 319, instead. If implemented as ECG electrodes, sensing electrodes 309 may be transducers that can help sense an ECG signal, e.g., a 12-lead signal, or a signal from a different number of leads, especially if they make good electrical contact with the body of the patient and in particular with the skin of the patient. As with defibrillation electrodes 304, 308, the support structure can be configured to be worn by patient 382 so as to maintain sensing electrodes 309 on a body of patient 382. For example, sensing electrodes 309 can be attached to the inside of support structure 170 for making good electrical contact with the patient, similarly with defibrillation electrodes 304, 308.

Many alternative sensing electrodes 309 are also envisioned. For example, sensing electrodes 309 may further include a perfusion sensor, a pulse oximeter, a device for detecting blood flow (e.g., a Doppler device), a sensor for detecting blood pressure (e.g., a cuff), an optical sensor, illumination detectors and sensors perhaps working together with light sources for detecting color change in tissue, a motion sensor, a device that can detect heart wall movement, a sound sensor, a device with a microphone, an SpO2 sensor, and so on. In view of this disclosure, it will be appreciated that such sensors can help detect the patient's pulse, and can therefore also be called pulse detection sensors, pulse sensors, and pulse rate sensors.

In some embodiments, defibrillator 300 also includes a measurement circuit 320, as one or more of its working together with its sensors or transducers. Measurement circuit 320 senses one or more electrical physiological signals of the patient from sensor port 319, if provided. Even if defibrillator 300 lacks sensor port 319, measurement circuit 320 may optionally obtain physiological signals through nodes 314, 318 instead, when defibrillation electrodes 304, 308 are attached to the patient. In these cases, the input reflects an ECG measurement. The patient parameter can be an ECG, which can be sensed as a voltage difference between electrodes 304, 308. In addition, the patient parameter can be an impedance, which can be sensed between electrodes 304, 308 and/or between the connections of sensor port 319 considered pairwise. Sensing the impedance can be useful for detecting, among other things, whether these electrodes 304, 308 and/or sensing electrodes 309 are not making good electrical contact with the patient's body. These patient physiological signals may be sensed when available. Measurement circuit 320 can then render or generate information about them as inputs, data, other signals, etc. As such, measurement circuit 320 can be configured to render a patient input responsive to a patient parameter sensed by a sensor. In some embodiments, measurement circuit 320 can be configured to render a patient input, such as values of an ECG signal, responsive to the ECG signal sensed by sensing electrodes 309. More strictly speaking, the information rendered by measurement circuit 320 is output from it, but this information can be called an input because it is received as an input by a subsequent device or functionality.

Defibrillator 300 also includes a processor 330. Processor 330 may be implemented in a number of ways in various embodiments. Such ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 330 may include, or have access to, a non-transitory storage medium, such as memory 338 that is described more fully later in this document. Such a memory can have a non-volatile component for storage of machine-readable and machine-executable instructions. A set of such instructions can also be called a program. The instructions, which may also be referred to as “software,” generally provide functionality by performing acts, operations and/or methods as may be disclosed herein or understood by one skilled in the art in view of the disclosed embodiments. In some embodiments, and as a matter of convention used herein, instances of the software may be referred to as a “module” and by other similar terms. Generally, a module includes a set of the instructions so as to offer or fulfill a particular functionality. Embodiments of modules and the functionality delivered are not limited by the embodiments described in this document.

Processor 330 can be considered to have a number of modules. One such module can be a detection module 332. Detection module 332 can include a Ventricular Fibrillation (VF) detector. The patient's sensed ECG from measurement circuit 320, which can be available as inputs, data that reflect values, or values of other signals, may be used by the VF detector to determine whether the patient is experiencing VF. Detecting VF is useful because VF typically results in SCA. Detection module 332 can also include a Ventricular Tachycardia (VT) detector, and so on.

Another such module in processor 330 can be an advice module 334, which generates advice for what to do. The advice can be based on outputs of detection module 332. There can be many types of advice according to embodiments. In some embodiments, the advice is a shock/no shock determination that processor 330 can make, for example via advice module 334. The shock/no shock determination can be made by executing a stored Shock Advisory Algorithm. A Shock Advisory Algorithm can make a shock/no shock determination from one or more ECG signals that are captured according to embodiments and determine whether or not a shock criterion is met. The determination can be made from a rhythm analysis of the captured ECG signal or otherwise.

In some embodiments, when the determination is to shock, an electrical charge is delivered to the patient. Delivering the electrical charge is also known as discharging and shocking the patient. As mentioned above, such can be for defibrillation, pacing, and so on.

In ideal conditions, a very reliable shock/no shock determination can be made from a segment of the sensed ECG signal of the patient. In practice, however, the ECG signal is often corrupted by electrical noise, which makes it difficult to analyze. Too much noise sometimes causes an incorrect detection of a heart arrhythmia, resulting in a false alarm to the patient. Noisy ECG signals may be handled as described in U.S. patent application Ser. No. 16/037,990, filed on Jul. 17, 2018, and since published as US 2019/0030351 A1, and also in U.S. patent application Ser. No. 16/038,007, filed on Jul. 17, 2018, and since published as US 2019/0030352 A1, both by the same applicant and incorporated herein by reference.

Processor 330 can include additional modules, such as other module 336, for other functions. In addition, if internal monitoring device 381 is indeed provided, processor 330 may receive its inputs, etc.

Defibrillator 300 optionally further includes a memory 338, for use by the processor 330 in conjunction with executing the several executable modules. Memory 338 may be implemented in a number of ways. Such ways include, by way of example and not of limitation, volatile memories, Nonvolatile Memories (NVM), Read-Only Memories (ROM), Random Access Memories (RAM), magnetic disk storage media, optical storage media, smart cards, flash memory devices, any combination of these, and so on. Memory 338 is thus a non-transitory storage medium. Memory 338, if provided, can include programs for processor 330, which processor 330 may be able to read and execute. More particularly, the programs can include sets of instructions in the form of code, which processor 330 may be able to execute upon reading. The programs may also include other information such as configuration data, profiles, scheduling etc. that can be acted on by the instructions. Executing is performed by physical manipulations of physical quantities, and may result in functions, operations, processes, acts, actions and/or methods to be performed, and/or the processor to cause other devices or components or blocks to perform such functions, operations, processes, acts, actions and/or methods. The programs can be operational for the inherent needs of processor 330 and can also include protocols and ways that decisions can be made by advice module 334. In addition, memory 338 can store prompts for user 382 if this user is a local rescuer. Moreover, memory 338 can store data. This data can include patient data, system data, and environmental data, for example as learned by internal monitoring device 381 and outside monitoring device 180. The data can be stored in memory 338 before it is transmitted out of defibrillator 300 or be stored there after it is received by defibrillator 300.

Defibrillator 300 can optionally include a communication module 390, for establishing one or more wired or wireless communication links with other devices of other entities, such as a mobile companion device, a remote assistance center, Emergency Medical Services (EMS), and so on. The communication links can be used to transfer data and commands. The data may be patient data, event information, therapy attempted, CPR performance, system data, environmental data, and so on. For example, communication module 390 may transmit wirelessly, e.g., on a daily basis, heart rate, respiratory rate, and other vital signs data to a server accessible over the internet, for instance as described in US 20140043149. This data can be analyzed directly by the patient's physician and can also be analyzed automatically by algorithms designed to detect a developing illness and then notify medical personnel via text, email, phone, etc. Communication module 390 may also include such interconnected sub-components as may be deemed necessary by a person skilled in the art, for example an antenna, portions of a processor, supporting electronics, outlet for a telephone or a network cable, etc.

Defibrillator 300 may also include a power source 340. To enable portability of defibrillator 300, power source 340 typically includes a battery. Such a battery is typically implemented as a battery pack, which can be rechargeable or not. Sometimes a combination is used of rechargeable and non-rechargeable battery packs. Other embodiments of power source 340 can include an AC power override, for where AC power will be available, an energy-storing capacitor, and so on. Appropriate components may be included to provide for charging or replacing power source 340. In some embodiments, power source 340 is controlled and/or monitored by processor 330.

Defibrillator 300 may additionally include an energy storage module 350. Energy storage module 350 can be coupled to the support structure of the WCD system, for example either directly or via the electrodes and their leads. Module 350 is where some electrical energy can be stored temporarily in the form of an electrical charge, when preparing it for discharge to administer a shock. In embodiments, module 350 can be charged from power source 340 to the desired amount of energy, as controlled by processor 330. In typical implementations, module 350 includes a capacitor 352, which can be a single capacitor or a system of capacitors, and so on. In some embodiments, energy storage module 350 includes a device that exhibits high power density, such as an ultracapacitor. As described above, capacitor 352 can store the energy in the form of an electrical charge, for delivering to the patient.

A decision to shock can be made responsive to the shock criterion being met. When the decision is to shock, processor 330 can be configured to cause at least some or all of the electrical charge stored in module 350 to be discharged through patient 182 while the support structure is worn by patient 182, so as to deliver a shock 111 to patient 182. For causing the discharge, defibrillator 300 moreover includes a discharge circuit 355. When the decision is to shock, processor 330 can be configured to control discharge circuit 355 to discharge through the patient at least some of all of the electrical charge stored in energy storage module 350. Discharging can be to nodes 314, 318, and from there to defibrillation electrodes 304, 308, so as to cause a shock to be delivered to the patient. Circuit 355 can include one or more switches 357. Switches 357 can be made in a number of ways, such as by an H-bridge, and so on. Circuit 355 could also be thus controlled via processor 330, and/or user interface 380. A time waveform of the discharge may be controlled by thus controlling discharge circuit 355. The amount of energy of the discharge can be controlled by how much energy storage module has been charged, and also by how long discharge circuit 355 is controlled to remain open.

Optionally, a WCD system according to embodiments also includes a fluid that it can deploy automatically between the defibrillation electrodes and the patient's skin. The fluid can be conductive, such as by including an electrolyte, for establishing a better electrical contact between the electrodes and the skin. Electrically speaking, when the fluid is deployed, the electrical impedance between each electrode and the skin is reduced. Mechanically speaking, the fluid may be in the form of a low-viscosity gel, so that it does not flow away, after being deployed, from the location it is released near the electrode. The fluid can be used for defibrillation electrodes 304, 308, and/or for sensing electrodes 309.

The fluid may be initially stored in a fluid reservoir, not shown in FIG. 3. Such a fluid reservoir can be coupled to the support structure. In addition, a WCD system according to embodiments further includes a fluid deploying mechanism 374. Fluid deploying mechanism 374 can be configured to cause at least some of the fluid to be released from the reservoir and be deployed near one or both of the patient locations to which electrodes 304, 308 are configured to be attached to the patient. In some embodiments, fluid deploying mechanism 374 is activated prior to the electrical discharge responsive to receiving activation signal AS from a processor 330, which is described more fully later in this document.

### Systems for Detecting Declines in Health and Probability of Adverse Events

Described here are specific systems and devices that may be used in various embodiments for detecting declines in a patient's health and for analyzing a probability that adverse events may be soon to occur.

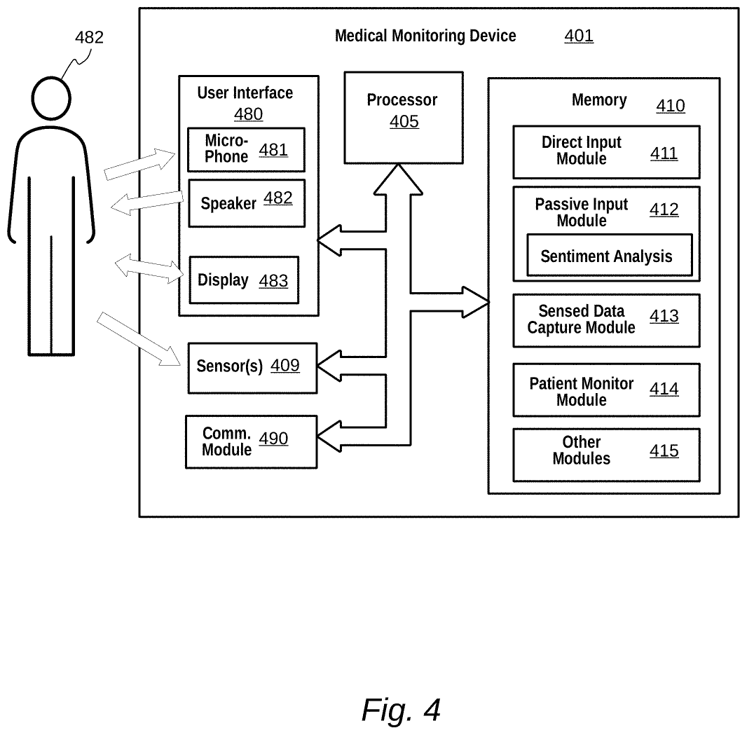

FIG. 4 is a functional block diagram generally illustrating a medical monitoring device 401 used in implementations of the disclosure. In some embodiments, medical monitoring device 401 may be implemented within or as part of the external defibrillator of a WCD system (e.g., external defibrillator 100, 200, or 300). Alternatively, the medical monitoring device 401 may be implemented within or as part of a standalone medical monitoring device (e.g., outside monitoring device 180). In another alternative, the medical monitoring device 401 may be implemented within or as part of a mobile companion device (e.g., companion device 199). Although illustrated in FIG. 4 as a unitary design, it will be appreciated that one or more of the functional blocks illustrated in FIG. 4 may, in various embodiments, be implemented within one or more other devices which are together incorporated into a WCD system.

The medical monitoring device 401 shown in FIG. 4 is shown, functionally, using a basic computing architecture that includes a processor 405, a memory 410, and a bus 412 that connects the processor 405 to the memory 410. In various embodiments, the processor 405 may be implemented as any form of instruction processing unit, such as a Central Processing Unit (CPU), a Graphics Processing Unit (GPU), a Data Processing Unit (DPU), an Accelerated Processing Unit (APU), a Tensor Processing Unit (TPU), or the like.

A user interface 480 is also included and is a logical component that includes features to enable a user (e.g., patient 482) to provide input to and receive output from the medical monitoring device 401. In various embodiments, the user interface 480 receives manual input from the patient (e.g., patient input such as a “health” rating). For example, the user interface 480 may include a microphone 481 to receive sounds and convert those sounds into computer-usable signals; the user interface 480 may also include a speaker 482 to convert computer-usable signals into sounds that may be heard; and the user interface 480 may include a display 483 to convert computer-usable signals into visual data that may be visually perceived. In preferred embodiments, the display 483 is a touchscreen display that may also receive tactile input in the form of touches on the display 483.

One or more sensors 420 may also be included in the medical monitoring device 401 for sensing physiological signals of the patient. As with the external defibrillator 300 shown in FIG. 3 and described above, the sensors 420 may include any one or more components used to detect various parameters (e.g., patient parameters, system parameters, environmental parameters, or the like). For instance, in some embodiments where the medical monitoring device 401 is implemented as part of an external defibrillator (e.g., external defibrillator 300) or external monitoring device (e.g., outside monitoring device 180), the sensors may include any or all of the sensors described above. In some embodiments where the medical monitoring device 401 is implemented within or as part of a companion device 199, the sensors may omit those used to capture patient parameters (e.g., ECG electrodes) and instead include only sensors used to detect environmental parameters (e.g., motion detector, accelerometer, compass, proximity sensor, barometer, or the like). It will be appreciated that in some embodiments, if the medical monitoring device 401 is implemented within a companion device 199, it could rely on sensor data collected by either or both of the external defibrillator or the outside monitoring device. In such embodiments, most or even all of the sensors 409 could omitted from or unused by the medical monitoring device 401 itself Still further, duplicative sensors (e.g., between the external defibrillator and the medical monitoring device 401) are also possible.

In other embodiments, the sensors 420 of the medical monitoring device 401 include one or more accelerometers and a walking detector, such as is disclosed in U.S. Published Patent Application No. 20190209853 entitled “Detecting Walking in a Wearable Cardioverter Defibrillator System, filed Oct. 11, 2018, and incorporated herein by reference in its entirety for all purposes. In such embodiments, the medical monitoring device 401 may capture the accelerometer and walking detector signals while the patient 482 is walking to determine the patient's gait.

A communication module 490 is also included in the medical monitoring device 401 to enable communication between the medical monitoring device 401 and other devices. In various embodiments, the communication module 490 may implement wired and/or wireless communication between the medical monitoring device 401 and an external defibrillator. For example, if the medical monitoring device 401 is implemented within a companion device (e.g., companion device 199), the communication module may enable bidirectional communication between the companion device 199 and the external defibrillator 100. In this way, the medical monitoring device 401 may receive signals from the external defibrillator 100, such as ECG waveforms and other patient parameters detected by the external defibrillator 100. Similarly, the medical monitoring device 401 may transmit instructions and/or data to the external defibrillator 100, such as a request for the external defibrillator 100 to transmit information to the medical monitoring device 401. Still further, the communication module 490 may implement cellular communications functionality to enable long-range cellular data communications with remote computing devices. These and other embodiments of the communication module 490 will be apparent to those skilled in the art.

A memory 410 is implemented within the medical monitoring device 401 to store various executable components and data. The memory 410 may be implemented as either volatile, non-volatile, or a combination of volatile and non-volatile memory. Non-volatile memory may be used to persistently store information, and volatile memory may be used by the processor 405 while executing various instructions. Accordingly, the term “memory” as used herein should be given its broadest interpretation as any repository in which computer-readable information may be held temporarily and/or permanently.

Within the memory 410 are several executable modules according to various embodiments. For example, a direct input module 411 may be included to receive and process direct user data provided by external sources. In one example, the direct input module 411 is configured to prompt the patient 482 for and accept data through the user interface 480, either through the microphone, the display, or both. The direct input module 411 may be configured to receive user-provided data and combine that data with other data, such as patient parameter data. For example, the patient 482 may be prompted to provide certain information by speaking to the microphone 481. Similarly, the patient 482 may be presented with choices that may be selected on the display 483. These are but a few examples of direct patient input.

A passive input module 412 may also be included to receive and process user data that is captured passively without necessarily prompting the patient 482 for input. For example, the passive input module 412 may be configured to monitor user speech, ambient sound, or both (collectively “passive sound”) received through the microphone 481 and to analyze that passive sound to assess and identify or estimate the patient's current subjective health level. The passive input module 412 may further include “sentiment analysis” functionality to analyze text recognized from the patient's speech to determine, generally, whether the patient's speech reveals that the patient is in a “good mood” or a “bad mood,” colloquially. Sentiment analysis is known in the art and need not be described in greater detail here.

Also, within memory 410 may be a sensed data capture module 413 configured to capture patient parameter output from the sensors 409 during a period that corresponds to the patient input period. By way of explanation, sensors 409 may be constantly capturing patient parameter data, such as the case when the patient 482 is wearing a WCD. However, in accordance with embodiments of the disclosure, it is advantageous to correlate the patient's objective physiological condition (as detected by the sensors 409) with the subjective health assessment provided by the patient (via the user interface 480) or derived from the patient's activities at the time the patient provides that health assessment. In other words, to improve adverse event prediction, it is most helpful to know the patient's objective health at the same moment the system learns the patient's subjective health.

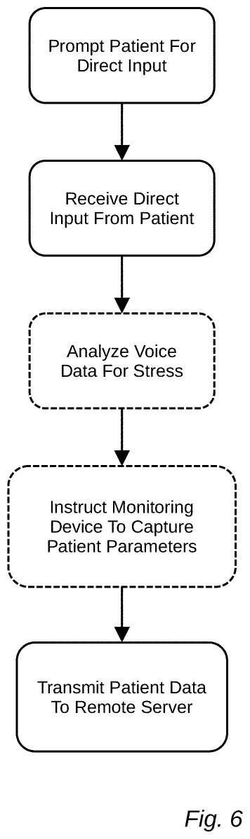

A patient monitor module 414 is also provided in the memory 410 to process the sensed data to implement the functionality of the medical monitoring device 401 as described herein. Particular techniques and functions that may be implemented within the conceptual patient monitor module 414 are described in greater detail below in conjunction with the techniques illustrated in FIGS. 6-9.

As discussed, the various components illustrated for convenience within medical monitoring device 480 may alternatively be implemented within or distributed across other devices. For example, in other embodiments the user interface 480 may be implemented in a companion mobile device (not shown) rather than in the medical monitoring device 480. In such an embodiment, the medical monitoring device 480 may employ the communication module 490 to communicate patient parameters and/or sensed data to the companion mobile device. The companion mobile device could then perform the function of prompting the patient to input the patient's health assessment. The companion device could then return that patient input and a “capture” signal to the medical monitoring device via the communication module 490 to cause the sensed data capture module 413 to initiate capture of the patient data, or the like. Many other permutations will be apparent to those skilled in the art.

The functional block diagram shown in FIG. 4 omits other components of the medical monitoring device 480 that are not directly involved in patient self-reported health rating functionality to avoid obscuring the description of this functionality.

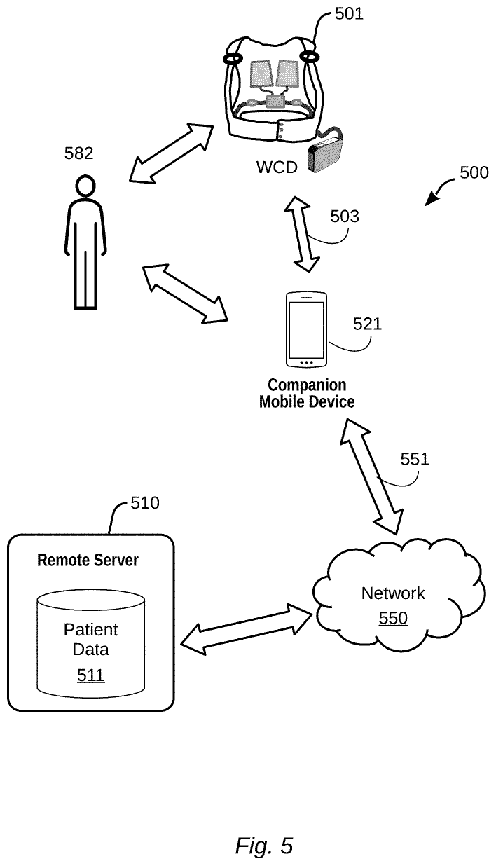

FIG. 5 is a conceptual diagram generally illustrating a health monitoring environment 500 according to embodiments. As illustrated in FIG. 5, the health monitoring environment 500 includes a wearable cardiac monitoring device (WCD 501), a mobile device 521, and a remote patient data platform (CareStation server 510). Each of those components variously communicates with one or more of the others either locally over a local communication link or remotely over a remote communication link through a wide area network 550, such as the Internet.

The wearable cardiac monitoring device 501 may be any medical device configured to detect and report on patient physiological parameters, such as described at length above. The wearable cardiac monitoring device 501 is described herein as a WCD for simplicity of discussion only. One example of such a WCD is the Assure WCD developed and offered by Kestra Medical Technologies, Inc. of Kirkland, Wash. Many other types of wearable cardiac monitoring devices may be used in various alternative embodiments without departing from the spirit of the disclosure. Accordingly, reference to use of a WCD as the cardiac monitoring device 501 is illustrative only and is not limiting of the disclosure.

The WCD 501 may also communicate over a local communication link 503 with a mobile device 521 operating an app configured to facilitate communication between the patient 582, the WCD 501, and other remote devices. In various embodiments, the mobile device 521 may be referred to as a companion device. In one example, the mobile device 521 and the WCD 501 may communicate using a relatively short-range local communication link 503, such as Ethernet, Bluetooth, or Wi-Fi. The mobile device 521 may also communicate with other remote devices using a remote communication link 551 to a wide area network 550, such as the Internet. In one specific embodiment, the application operating on the mobile device 521 may be the Assure patient app developed and offered by Kestra Medical Technologies, Inc. of Kirkland, Wash. In various embodiments, the patient application operating on the mobile device 521 may provide a graphical user interface (GUI) that enables review of patient physiological parameters captured by the WCD 501.

In a preferred embodiment, the remote patient data platform (CareStation server 510) is implemented as a remote server for use by medical professionals and/or clinicians that offers efficient tools for managing cardiac patient care. In various embodiments, the remote patient data platform delivers relevant data and valuable insights into patient heart rhythms and usage compliance by providing clear patient reports that include VT, VF, bradycardia, asystole, and non-sustained ventricular arrhythmia episodes; WCD usage and physical activity trends; and may include a population dashboard with configurable notifications. One example of such a remote patient data platform is the CareStation platform developed and offered by Kestra Medical Technologies, Inc. of Kirkland, Washington. The remote patient data platform is described herein as a CareStation server for simplicity of discussion only. Many other types of remote patient data platforms may be used in various alternative embodiments without departing from the spirit of the disclosure. Accordingly, reference to use of a CareStation server as the remote patient data platform is illustrative only and is not limiting of the disclosure.

Generally stated, patient data is collected by the WCD 501 and uploaded, either by the WCD 501 directly or by using an associated mobile device (e.g., companion device 521), to the CareStation server 510. In addition, the mobile device 521 may also collect some forms of patient data. In various embodiments, the patient data includes both patient physiological data (e.g., ECG waveforms, and the like) and patient health assessment rating data. As discussed above, in various embodiments, the patient 582 may provide a health assessment rating, either directly or passively, which represents a subjective measure of how well the patient feels. The patient physiological data is correlated with the patent health assessment rating and stored as patient data 511 at the CareStation server 510. In various embodiments, either or both of the mobile device 521 or the WCD 501 may collect the physiological data and the patient health assessment rating data as described above.

The CareStation server 510 stores the patient data and may perform several analyses on the patient data to identify patient health issues, such as the occurrence of arrythmias, shockable and non-shockable events, and other medical events. In addition, after-action evaluations may be performed on the patient data to help improve the quality of future shock therapy.

In accordance with embodiments of the disclosure, the CareStation server 510 may also be configured to analyze patient physiological data and to correlate the patient parameter data with provided subjective health assessment rating provided by the patient 582. As noted above, an individual's personal assessment of his or her own health based on how well the person feels can be a useful tool when correlated with objective physiological indicators of that person's cardiac health. For example, in some cases a person may simply feel “bad” for some time (e.g., hours or days) prior to onset of a serious health event, such as SCA. That person's objective physiological data may not have revealed any significant indicators of an impending SCA which could have been used to alter treatment or provide a warning to the patient and to the patient's caregivers. However, by correlating and analyzing that person's subjective health assessment ratings that show the person “felt bad” with that person's objective physiological data, perhaps subtle indicators of an impending health event can be discerned. In this way, treatment for a patient can be enhanced and tailored not only to react to the onset of adverse health events, but also to hopefully predict imminent adverse health events before they occur.

In a further enhancement, patient data 511 may include data received from multiple different patients (the “patient population”). In such embodiments, one or more applications miming on the remote server 510 would aggregate the patient data 511 and use the aggregated patient data to learn relationships between trends in patient evaluation (self or sentiment predicted) and adverse events (SCD, AMI, Stroke, TIA, etc.). These embodiments could use the patient health rating data along with patient outcomes as a training set for a machine learning classifier. Once trained, the classifier would score new patient data for its likelihood of leading to an adverse event. For example, in WCD embodiments, episodes sourced by the WCDs would be used for the patient outcomes. To predict other adverse events, patient outcome data can be acquired from the care providers.

### Techniques for Detecting Declines in Health and Probability of Adverse Events

Described next are various techniques that may be implemented by preferred embodiments of the disclosure to collect patient health assessment ratings. These techniques may be embodied as processes and/or algorithms implemented in the various medical devices illustrated in FIGS. 1-5 and described above. Additional embodiments will also become apparent to those skilled in the art from the teachings of this disclosure.

### Direct Input Embodiments

FIG. 6 illustrates an illustrative flow diagram implementing a process 600 for receiving direct input from a patient. In this embodiment, a patient may (but need not) be prompted to directly provide the system with a health assessment rating.

At step 601, the patient is prompted to rate how the patient “feels” at that time. The prompt may take the form of a voice prompt, or it may be presented as selectable options on a touchscreen display. These are but illustrative examples and many other forms of prompt are possible. The prompt may ask the patient to provide a subjective assessment of the patient's current health (e.g., a health rating), such as on a fixed scale (e.g., 1-10 or 1-5) into the wearable medical device or companion device. Such prompt may be presented to the patient periodically, such as every day or at multiple times throughout the day.

At step 603, the patient inputs the health assessment rating to the system. In various embodiments, the input could take the form of a direct manual input, such as via touchscreen display or keyboard. In other embodiments, the patient could input the health assessment rating using voice input by speaking into a microphone. In such embodiments, the patient's voice input could be converted to numerical data on the fixed scale.

At optional step 605, in embodiments where the patient provides input by voice, the voice waveform could also be analyzed for signs of stress and changes in speech patterns. Such information could be analyzed either as a weighting factor for the patient's subjective health assessment, or it could be incorporated into the patient's objective physiological parameter data for later analysis.

At optional step 607, the system may issue an instruction to a medical monitoring device to capture patient parameter data, such as physiological data. This step may be necessary in embodiments where the patient's subjective health assessment rating is provided on a device which does not have sufficient sensors to capture the patient parameter data, such as a companion device. In such an embodiment, the companion device may issue an instruction to another medical device, such as a WCD or other medical monitoring device, to capture the patient parameter data, such as ECG waveform data and possibly other sensed patient parameters.

At step 609, the patient health assessment rating and the one or more sensed patient parameters (e.g., a patient's current vital signs and a brief ECG recording) are transmitted to a remote server for data aggregation and, in some embodiments, clinical review. The rating and the stress rating along with the patient's current vital signs and a brief ECG recording are transmitted to a remote server for data aggregation and possible clinical review.

### Passive Input Embodiments

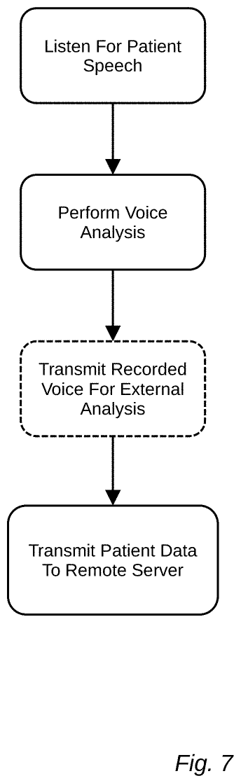

FIG. 7 illustrates an illustrative flow diagram implementing a process for receiving passive input from a patient. In such embodiments, the system may not prompt the patient to provide a health assessment rating. Instead, the system obtains such rating by analyzing the environment to detect health assessment criteria.

At step 701, the system listens to the patient's ambient speech, such as daily conversations, to capture voice data. In such embodiments, the voice data may be unprompted so that the patient need not take any special steps to input health assessment data. Indeed, the patient may not even be aware that the voice data is being collected at that time. To address privacy concerns, in some embodiments patient's conversation cannot be permanently saved. In many embodiments, the system prompts the patient to agree in advance to voice monitoring, perhaps for a defined period of time or until cancelled, before the system begins voice monitoring. The system may also be configured to periodically prompt the patient to agree or reaffirm that the system may monitor the patient's voice. In still other embodiments, the system prompts the patient with questions that the patient can answer by voice. Such embodiments have the benefit of creating additional voice data which may be analyzed as described below. The system may be configured with additional questions so that the patient will provide enough voice data for analysis. For example, such prompting would be tailored to avoid “yes or no” questions.

At step 703, the system performs a voice analysis on the captured voice data. In various embodiments, the analysis may take the form of a voice waveform analysis (e.g., stress analysis) to qualify the patient's current health as revealed through the patient's voice. In addition, the voice waveform(s) may be converted to a textual transcript of the patient's conversation(s) and the analysis may take the form of a sentiment analysis (Natural Language Processing) of the patient's conversations. One or more of these analyses may be used to generate a health assessment rating that estimates the current subjective health of the patient—how the patient “feels.”

In embodiments where the system prompts the patient for verbal consent to voice monitoring, the system may additionally analyze the patient's verbal response(s). In a further enhancement, in some embodiments the system can also analyze the voice waveform for slurred speech, which could be an indicator of stroke or transient ischemic attack (TIA).

At optional step 705, the captured voice waveforms may be transmitted to an external computing system, such as a remote server, for analysis. This step may be performed, for example, in embodiments where the device on which the voice data is captured may not have sufficient processing power or other resources to adequately execute the appropriate analysis. In such cases, the data may be transmitted to an external server (e.g., CareStation server 510) for analysis. In some embodiments, the sentiment analysis could be run on the wearable medical device or the companion mobile device. In other embodiments, the wearable medical device and/or the companion mobile device includes network connectivity (e.g., cellular data, Wi-Fi, etc.), and can be configured to have the sentiment analysis be performed on an external server.

At step 707, the system transmits data to an external server for further analysis and clinical review. The data transmitted may include the voice recording, voice-to-text result, or stress analysis results, sentiment analysis results, and patient sensed physiological data (e.g., current vital signs and a brief ECG recording).

Unlike the direct input embodiments where the exact time that the patient provides input is fixed, the passive input embodiments may collect data over an extended period of time. Accordingly, correlating the patient sensed physiological data with the passive input data may be less precise. However, correlating the passive input with the sensed parameter data is still possible and preferred.

Some such embodiments can be advantageous over other systems. For example, patients can be in denial of their symptoms and not report how they truly feel to a loved one or care giver. The words they choose in conversations, however, may indicate a change in mood or condition, which can be detected in such embodiments and used to predict or recognize declines in the patient's health.

### Patient Gait Embodiments

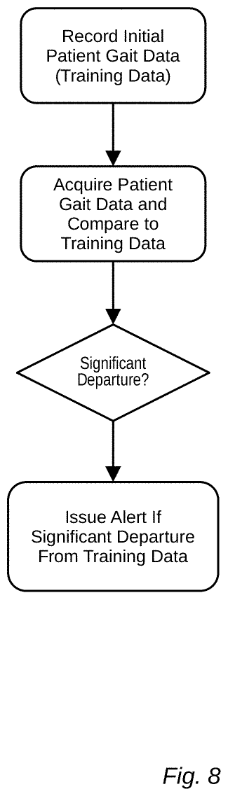

FIG. 8 is a flow diagram generally illustrating another form of passive input embodiment that analyzes a patient's gait to determine a health assessment rating. Similar to how the patient's subjective assessment of how the patient feels may be useful for recognizing or predicting adverse events or significant declines in health, analyzing changes in the patient's gait, either alone or in combination with any one or more of the above-described techniques, may be used to recognize or predict adverse events or significant declines in health. Changes in gait can have many causes, such as injury or neurodegeneration (stroke, Alzheimer's, Parkinson's, MS, ALS, etc.) just to name a few. Falls could be due to similar causes as gait changes, accidents or cardiac events. The patient gait embodiments may be implemented alone or combined with other embodiments.

In patient gait embodiments, a process 800 may be executed by one or more applications distributed over one or more of the medical monitoring devices 401 and/or the remote server 510 and configured to perform the following operations.

At step 801, the process 800 records and stores the patient's initial accelerometer signals while walking during a training period, such as the first day(s) of patient wear. The training period provides a reference measurement. Once training is sufficiently complete, the process begins the gait analysis phase.

At step 803, the process 800 (1) acquires the walking accelerometer signals (current measurement), and (2) compares the current measurement with the reference measurement to determine if there has been a significant change in the patient's gait, such as a degradation in the patient's balance or stride pattern indicating a need for evaluation of a possible change in patient's ability to balance. Specific examples of such gait analysis include (1) determining differences in step length, (2) determining differences in individual leg stride, (2) determining the amount of time spent on both feet between steps, (3) determining changes in patient posture (or lean), (4) detecting patient falls through analysis of the accelerometer Acceleration and Jerk signals, and (5) determining differences in walking speed.

If the process 800 identifies any significant change in the patient's gait, it will record the time of the detected event, correlated accelerometer waveforms, and any other available vital signs and waveforms.

At step 805, if a significant change between the reference measurement and the current measurement is detected, the process 800 issues an alert to notify medical care givers of potential need for follow up due to significant changes in gate or balance or if a fall has been detected. The alert may be issued directly to the patient, to a remote system (e.g., CareStation 510), to both, and/or to any other caregiver for the patient.

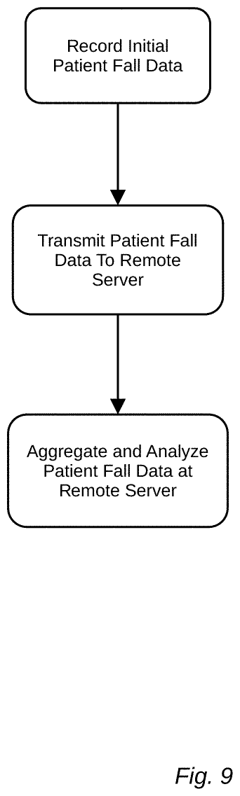

FIG. 9 is a functional flow diagram generally illustrating an enhanced process 900 that may be implemented in systems that have a gyroscope, in addition to one or more accelerometers, and are configured to detect patient falls.

At step 901, the process 900 detects and records any accelerometer signals, gyroscope signals, and patient fall events that may have been encountered by the medical monitoring device associated with the patient.

At step 903, the process 900 communicates the accelerometer signals, gyroscope signals, and any patient fall events to a remote server, which are then stored for analysis.

At step 905, the stored accelerometer, gyroscope, and patient fall data are aggregated and analyzed using machine learning algorithms and integrated into the process 800, where appropriate, to predict patients who are at enhanced risk of falling soon.

### Other Alternative Embodiments

In some embodiments, various features of the above-described embodiments are combined. For example, some embodiments can prompt the user to directly enter a health assessment rating while also performing an analysis of the patient's speech as described above. In other embodiments, the system can prompt the user for a health assessment rating via voice input and analyze the patient's speech as described above.

In still other embodiments, the system can add the patient taking a digital image of his or her face in combination with any of the previously described embodiments. In such embodiments, the digital image could then be packaged with the other data and waveforms that are uploaded to the remote server. The image could then be analyzed for signs of facial paralysis (indicating possible TIA or stroke) on the remote server. The remote server can then make the results of the analysis available to authorized users such as, for example, the patient's physician.

Other embodiments include combinations and sub-combinations of features described or shown in the drawings herein, including for example, embodiments that are equivalent to providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, feature or features can refer to the structures and/or functions of an apparatus, article of manufacture or system, and/or the steps, acts, or modalities of a method.

The embodiments of the disclosure in which an exclusive property or privilege is claimed are recited as follows:

1. A medical monitoring device for a patient, comprising:
a user interface configured to receive input from the patient;
a sensed data capture module configured to record at least a portion of patient parameter data, the patient parameter data including at least physiological data of the patient; and
a patient monitor module configured to receive the input from the user interface, and further configured to receive the portion of patient parameter data from the sensed data capture module, the patient monitor module being further configured to temporally correlate the input from the user interface with the portion of patient parameter data.

2. The medical monitoring device recited in claim 1, wherein the input comprises manually input data, the manually input data further comprising a health assessment rating that represents how the patient subjectively feels on a scale.

3. The medical monitoring device recited in claim 1, wherein the input comprises audibly input data that represents how the patient subjectively feels.

4. The medical monitoring device recited in claim 3, wherein the audibly input data comprises a direct indication of how the patient subjectively feels on a scale.

5. The medical monitoring device recited in claim 3, wherein the audibly input data comprises an audio recording of the patient after the patient has been prompted to provide the audible input data.

6. The medical monitoring device recited in claim 3, wherein the audibly input data comprises an audio recording of the patient passively captured.

7. The medical monitoring device recited in claim 1, further comprising a communication module configured to enable bidirectional communication between the medical monitoring device and a remote server, the patient monitor module being further configured to transmit at least the correlated portion of patient parameter data to the remote server using the communication module.

8. The medical monitoring device recited in claim 7, wherein the sensed data capture module is further configured to receive, via the communication module, the patient parameter data from a wearable medical device that includes sensors to capture the patient parameter data.

9. The medical monitoring device recited in claim 1, further comprising one or more sensors configured to capture the patient parameter data, and wherein the sensed data capture module is further configured to receive the patient parameter data from the one or more sensors.

10. The medical monitoring device recited in claim 9, wherein the medical monitoring device comprises a Wearable Cardioverter Defibrillator (WCD).

11. The medical monitoring device recited in claim 3, wherein the medical monitoring device further comprises a sentiment analysis module to analyze the audibly input data to identify a health assessment rating for how the patient subjectively feels.

12. The medical monitoring device recited in claim 3, wherein the patient monitor module is further configured to analyze the audibly input data to identify a stress level of the patient.

13. The medical monitoring device for a patient, comprising:
a remote server configured to receive data over a communication link to a wide area network, the remote server being configured to receive a health assessment rating and patient parameter data, the health assessment rating being temporally correlated to the patient parameter data, the remote server being further configured to analyze the correlated health assessment rating and patient parameter data to identify indicia in the patient parameter data that indicates the presence of a heightened risk that an adverse health event will occur.

14. The medical monitoring device recited in claim 13, wherein the remote server receives the data from a companion device associated with the patient.

15. The medical monitoring device recited in claim 13, wherein the remote server receives the data from a Wearable Cardioverter Defibrillator.

16. The medical monitoring device recited in claim 13, wherein the remote server receives the data from an external monitoring device associated with a Wearable Cardioverter Defibrillator system.

17. The medical monitoring device recited in claim 13, wherein the remote server is further configured to aggregate data for multiple different patients and to analyze the aggregated data to identify the indicia of an adverse health event.

18. A method of predicting the occurrence of an adverse health event, comprising:
receiving patient input tending to indicate a subjective quality of health for the patient;
receiving patient parameter data that shows objective physiological criteria for the patient, the physiological criteria including an electrocardiogram waveform;
correlating the patient input with the patient parameter data;
analyzing the patient parameter data to identify indicia of the adverse health event; and
analyzing additional patient parameter data to identify the indicia of the adverse health event.

19. The method recited in claim 18, further comprising analyzing additional patient parameter data associated with an occurrence of the adverse health event to investigate whether the indicia of the adverse health event was present prior to the adverse health event.

20. The method recited in claim 18, further comprising issuing an alert if the indicia of the adverse health event is present in the additional patient parameter data.
